(19) United States
(12) Patent Application Publication
(10) Pub. No.: US 2019/0314617 A1
(43) Pub. Date: Oct. 17, 2019

(54) METHOD FOR INHIBITING AIR BUBBLES ON AN INFLATABLE BALLOON OF AN INTRAVASCULAR BALLOON CATHETER SYSTEM

(71) Applicant: Cryterion Medical, Inc., Carlsbad, CA (US)

(72) Inventor: Chadi Harmouche, Quebec (CA)

(21) Appl. No.: 16/384,596

(22) Filed: Apr. 15, 2019

Related U.S. Application Data
(60) Provisional application No. 62/658,242, filed on Apr. 16, 2018.

(51) Int. Cl. A61M 25/10; A61F 7/12

(52) U.S. Cl. CPC A61M 25/10184; A61M 25/10181; A61F 2007/0056; A61M 2025/1077; A61F 7/123

(57) ABSTRACT

A method for conditioning an inflatable balloon of a balloon catheter prior to use inside a patient includes submerging the inflatable balloon in a liquid solution, inflating the inflatable balloon, removing any bubbles on an exterior surface of the inflated, inflatable balloon, deflating the inflatable balloon while maintaining the inflatable balloon submerged in the liquid solution, and retracting the deflated, inflatable balloon into a catheter sheath while maintaining the inflatable balloon submerged in the liquid solution.

### CROSS-REFERENCE TO RELATED APPLICATION

This application claims priority to Provisional Application No. 62/658,242, filed Apr. 16, 2018, which is herein incorporated by reference in its entirety.

### TECHNICAL FIELD

The present disclosure relates to medical devices and methods for treating cardiac arrhythmias. More specifically, the disclosure relates to devices and methods for cardiac cryoablation.

### BACKGROUND

Cardiac arrhythmias involve an abnormality in the electrical conduction of the heart and are a leading cause of stroke, heart disease, and sudden cardiac death. Treatment options for patients with arrhythmias include medications and/or the use of medical devices, which can include implantable devices and/or catheter ablation of cardiac tissue, to name a few. In particular, catheter ablation involves delivering ablative energy to tissue inside the heart to block aberrant electrical activity from depolarizing heart muscle cells out of synchrony with the heart's normal conduction pattern. The procedure is performed by positioning the tip of an energy delivery catheter adjacent to diseased or targeted tissue in the heart. The energy delivery component of the system is typically at or near the most distal (i.e. farthest from the user or operator) portion of the catheter, and often at the tip of the catheter.

Various forms of energy can be used to ablate diseased heart tissue. These can include radio frequency (RF), cryogenics, ultrasound and laser energy, to name a few. During an ablation procedure, with the aid of a guide wire, the distal tip of the catheter is positioned adjacent to targeted cardiac tissue, at which time energy is delivered to create tissue necrosis, rendering the ablated tissue incapable of conducting electrical signals. The dose of the energy delivered is a critical factor in increasing the likelihood that the treated tissue is permanently incapable of conduction. At the same time, delicate collateral tissue, such as the esophagus, the bronchus, and the phrenic nerve surrounding the ablation zone can be damaged and can lead to undesired complications. Thus, the operator must finely balance delivering therapeutic levels of energy to achieve intended tissue necrosis while avoiding excessive energy leading to collateral tissue injury.

Atrial fibrillation (AF) is one of the most common arrhythmias treated using catheter ablation. In the earliest stages of the disease, paroxysmal AF, the treatment strategy involves isolating the pulmonary veins from the left atrial chamber. Recently, the use of techniques known as intravascular balloon catheter procedures to treat AF have increased. In part, this stems from the ease of use of the balloon catheters included in such systems, shorter procedure times and improved patient outcomes. More specifically, balloon catheters can include one or more inflatable/deflatable cryoballoons (or other types of balloons, generically referred to herein as “inflatable balloons”) that are positioned inside the cardiovascular (or circulatory) system of a patient.

Typical balloon catheters in a deflated state are folded during the manufacturing process. The folded, deflated balloon catheter is then introduced into a catheter sheath in order to facilitate insertion of the balloon catheter into the body of the patient. Unfortunately, air bubbles can be trapped in the folds of the balloon catheter. These unwanted air bubbles can lead to an air embolism, which can be injurious or even fatal to the patient.

### SUMMARY

Example 1 is a method for conditioning an inflatable balloon of a balloon catheter prior to use inside a patient. The method includes submerging the inflatable balloon in a liquid solution, inflating the inflatable balloon, removing any bubbles on an exterior surface of the inflated, inflatable balloon, deflating the inflatable balloon while maintaining the inflatable balloon submerged in the liquid solution, and retracting the deflated, inflatable balloon into a catheter sheath while maintaining the inflatable balloon, submerged in the liquid solution.

Example 2 is the method of Example 1, further comprising removing the sheathed, deflated, inflatable balloon from the liquid solution.

Example 3 is the method of Example 2, wherein submerging the inflatable balloon in the liquid solution includes submerging the inflatable balloon in the liquid solution with the balloon in a deflated state, and wherein inflating the inflatable balloon is performed after submerging the inflatable balloon in the liquid solution.

Example 4 is the method of Example 2, wherein inflating the inflatable balloon is performed prior to submerging the inflatable balloon in the liquid solution.

Example 5 is the method of any of Examples 2-4, wherein the balloon catheter is a cryoablation catheter, and wherein the inflatable balloon is configured to receive a cryogenic fluid.

Example 6 is the method of Example 5, wherein the inflatable balloon is a double-balloon structure having an outer balloon and an inner balloon disposed and contained within the outer balloon.

Example 7 is the method of Example 5 or Example 6, further comprising performing a cryoablation procedure after removing the sheathed, deflated, inflatable balloon from the liquid solution.

Example 8 is a method for conditioning an inflatable balloon of a balloon catheter prior to use inside a patient. The method includes the steps of positioning the balloon catheter within a catheter sheath such that the inflatable balloon extends from an end of the catheter sheath, submerging the end of the catheter sheath and the inflatable balloon in a liquid solution, inflating the inflatable balloon, removing any bubbles on an exterior surface of the inflated, inflatable balloon, deflating the inflatable balloon, and retracting the deflated, inflatable balloon into the catheter sheath while maintaining the inflatable balloon, the guidewire lumen and the end of the catheter sheath submerged in the liquid solution.

Example 9 is the method of Example 8, further comprising removing the catheter sheath and the deflated, inflatable balloon from the liquid solution.

Example 10 is the method of Example 9, wherein submerging end of the catheter sheath and the inflatable balloon in the liquid solution is performed with the inflatable balloon in a deflated state, and wherein inflating the inflatable balloon is performed after submerging the inflatable balloon in the liquid solution.

Example 11 is the method of Example 9, wherein inflating the inflatable balloon is performed prior to submerging the end of the catheter sheath and the inflatable balloon in the liquid solution.

Example 12 it the method of any of Examples 9-11, wherein the balloon catheter is a cryoablation catheter, and wherein the inflatable balloon is configured to receive a cryogenic fluid.

Example 13 is the method of Example 12, wherein the inflatable balloon is a double-balloon structure having an outer balloon and an inner balloon disposed and contained within the outer balloon.

Example 14 is the method of Example 12 or Example 13, further comprising performing a cryoablation procedure with the balloon catheter after removing the catheter sheath and the inflatable balloon from the liquid solution.

Example 15 is a method for conditioning an inflatable balloon of a balloon catheter prior to use inside a patient, the balloon catheter including a guidewire lumen. The method includes the steps of disposing the balloon catheter within a catheter sheath, with the inflatable balloon in a deflated state, submerging an end of the catheter sheath, the inflatable balloon and the guidewire lumen in a liquid solution, moving the balloon catheter longitudinally relative to the catheter sheath so that the inflatable balloon and the guidewire lumen extend from the end of the catheter sheath, inflating the inflatable balloon, removing any bubbles on an exterior surface of the inflated, inflatable balloon, deflating the inflatable balloon, and retracting the deflated, inflatable balloon and the guidewire lumen into the catheter sheath while maintaining the inflatable balloon, the guidewire lumen and the end of the catheter sheath submerged in the liquid solution.

Example 16 is the method of Example 15, further comprising removing the catheter sheath and the deflated, inflatable balloon from the liquid solution.

Example 17 is the method of Example 16, wherein the balloon catheter is a cryoablation catheter, and wherein the inflatable balloon is configured to receive a cryogenic fluid.

Example 18 is the method of Example 16 or Example 17, wherein the inflatable balloon is a double-balloon structure having an outer balloon and an inner balloon disposed and contained within the outer balloon.

Example 19 is the method of any of Examples 16-18, further comprising disposing a guidewire within the guidewire lumen prior to submerging the end of the catheter sheath, the inflatable balloon and the guidewire lumen in the liquid solution.

Example 20 is the method of any of Examples 16-19, further comprising performing a cryoablation procedure with the balloon catheter after removing the catheter sheath and the inflatable balloon from the liquid solution.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a simplified schematic side view illustration of a patient and one embodiment of an intravascular balloon catheter system having features of the present disclosure;

FIG. 2A is a simplified side view of a portion of one embodiment of the intravascular balloon catheter system including an inflatable balloon shown in a preconditioned, deflated state, with the inflatable balloon being submerged in a liquid solution, and a plurality of air bubbles on the inflatable balloon;

FIG. 2B is a simplified side view of the portion of the intravascular balloon catheter system illustrated in FIG. 2A, including the inflatable balloon shown in a conditioned, inflated state, with the inflatable balloon being submerged in a liquid solution;

FIG. 2C is a simplified side view of the portion of the intravascular balloon catheter system illustrated in FIG. 2A, including the inflatable balloon shown in a conditioned, deflated state; and

FIG. 3 is a flow chart providing one embodiment of a method for inhibiting the presence of air bubbles on the inflatable balloon of the intravascular balloon catheter system.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

Embodiments of the present disclosure are described herein in the context of a cryogenic balloon catheter system (also hereinafter sometimes referred to as an “intravascular catheter system”). Those of ordinary skill in the art will realize that the following detailed description of the present disclosure is illustrative only and is not intended to be in any way limiting. Other embodiments of the present disclosure will readily suggest themselves to such skilled persons having the benefit of this disclosure. Reference will now be made in detail to implementations of the present disclosure as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Although the disclosure provided herein can be applied to cryogenics, it is understood that various other forms of energy can be used to ablate diseased heart tissue. These can include radio frequency (RF), ultrasound and laser energy, as non-exclusive examples. The present disclosure is intended to be effective with any or all of these and other forms of energy that can be used with intravascular balloon catheters.

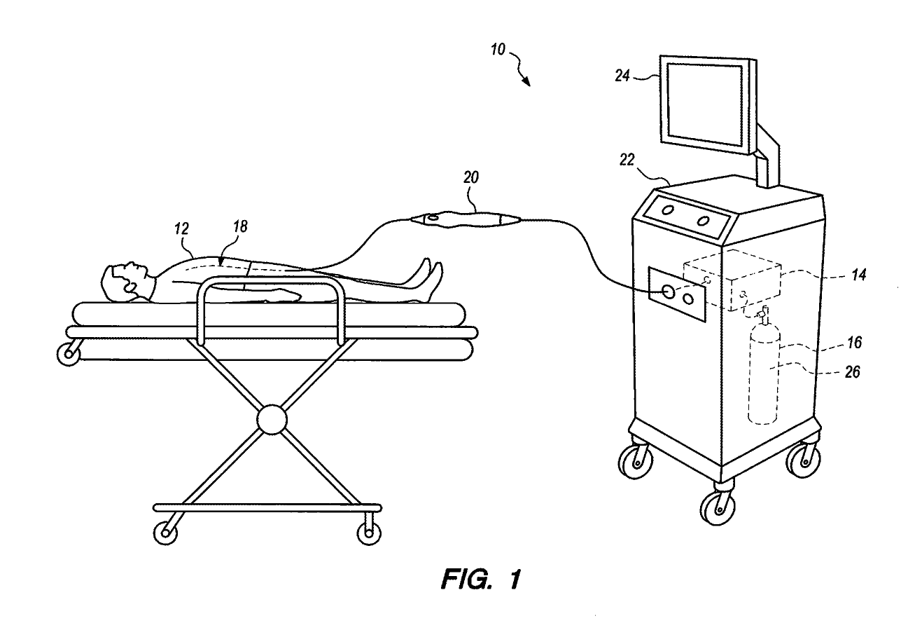

FIG. 1 is a schematic side view illustration of one embodiment of a medical device 10 for use with a patient 12, which can be a human being or an animal. Although the specific medical device 10 shown and described herein pertains to and refers to an intravascular balloon catheter system 10 (also sometimes referred to herein as a “catheter system”), it is understood and appreciated that other types of medical devices 10 can equally benefit by the teachings provided herein. The design of the catheter system 10 can be varied. In certain embodiments such as the embodiment illustrated in FIG. 1, the catheter system 10 can include one or more of a control system 14, a fluid source 16, a balloon catheter 18, a handle assembly 20, a control console 22 and a graphical display 24. It is understood that although FIG. 1 illustrates the structures of the catheter system 10 in a particular position, sequence and/or order, these structures can be located in any suitably different position, sequence and/or order than that illustrated in FIG. 1.

In various embodiments, the control system 14 can control release and/or retrieval of a cryogenic fluid 26 to and/or from the balloon catheter 18. In various embodiments, the control system 14 can control activation and/or deactivation of one or more other processes of the balloon catheter 18. Additionally, or in the alternative, the control system 14 can receive electrical signals, including data and/or other information (hereinafter sometimes referred to as “sensor output”) from various structures within the catheter system 10. In some embodiments, the control system 14 can assimilate and/or integrate the sensor output, and/or any other data or information received from any structure within the catheter system 10. Additionally, or in the alternative, the control system 14 can control positioning of portions of the balloon catheter 18 within the body of the patient 12, and/or can control any other suitable functions of the balloon catheter 18.

The fluid source 16 contains the cryogenic fluid 26, which is delivered to the balloon catheter 18 with or without input from the control system 14 during a cryoablation procedure. The type of cryogenic fluid 26 that is used during the cryoablation procedure can vary. In one non-exclusive embodiment, the cryogenic fluid 26 can include liquid nitrous oxide. However, any other suitable cryogenic fluid 26 can be used.

The balloon catheter 18 is inserted into the body of the patient 12. In one embodiment, the balloon catheter 18 can be positioned within the body of the patient 12 using the control system 14. Alternatively, the balloon catheter 18 can be manually positioned within the body of the patient 12 by a health care professional (also sometimes referred to herein as an “operator”). In certain embodiments, the balloon catheter 18 is positioned within the body of the patient 12 utilizing the sensor output from the balloon catheter 18. In various embodiments, the sensor output is received by the control system 14, which then can provide the operator with information regarding the positioning of the balloon catheter 18. Based at least partially on the sensor output feedback received by the control system 14, the operator can adjust the positioning of the balloon catheter 18 within the body of the patient 12. While specific reference is made herein to the balloon catheter 18, it is understood that any suitable type of medical device and/or catheter may be used.

The handle assembly 20 is handled and used by the operator to operate, position and/or control the balloon catheter 18. The design and specific features of the handle assembly 20 can vary to suit the design requirements of the cryogenic balloon catheter system 10. In the embodiment illustrated in FIG. 1, the handle assembly 20 is separate from, but in electrical and/or fluid communication with the control system 14, the fluid source 16 and/or the graphical display 24. In some embodiments, the handle assembly 20 can integrate and/or include at least a portion of the control system 14 within an interior of the handle assembly 20. It is understood that the handle assembly 20 can include fewer or additional components than those specifically illustrated and described herein.

In the embodiment illustrated in FIG. 1, the control console 22 includes the control system 14, the fluid source 16 and the graphical display 24. However, in alternative embodiments, the control console 22 can contain additional structures not shown or described herein. Still alternatively, the control console 22 may not include various structures that are illustrated within the control console 22 in FIG. 1. For example, in one embodiment, the control console 22 does not include the graphical display 24.

The graphical display 24 provides the operator of the catheter system 10 with information that can be used before, during and after the cryoablation procedure. The specifics of the graphical display 24 can vary depending upon the design requirements of the catheter system 10, or the specific needs, specifications and/or desires of the operator.

In one embodiment, the graphical display 24 can provide static visual data and/or information to the operator. In addition, or in the alternative, the graphical display 24 can provide dynamic visual data and/or information to the operator, such as video data or any other data that changes over time. Further, in various embodiments, the graphical display 24 can include one or more colors, different sizes, varying brightness, etc., that may act as alerts to the operator. Additionally, or in the alternative, the graphical display can provide audio data or information to the operator.

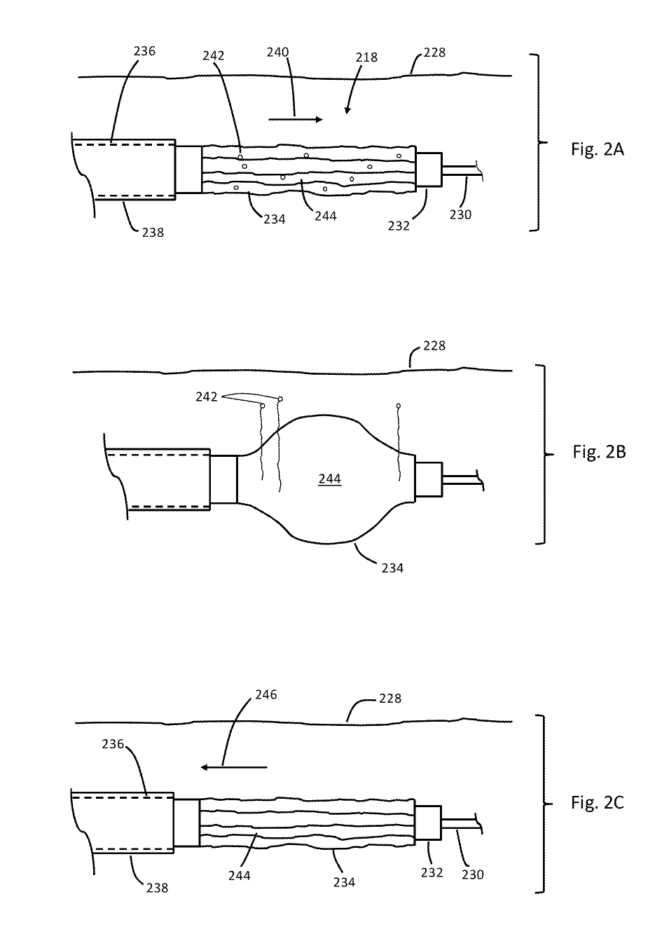

As an overview, FIGS. 2A-2C illustrate one or more embodiments of at least one sequence outlining a method that can be used prior to insertion of the balloon catheter into the vascular system of the patient and/or prior to use of the catheter system in the first instance. Stated another way, the sequence illustrated and/or described herein can be used prior to use of the balloon catheter, e.g., to “condition” the balloon catheter. By conditioning the balloon catheter as set forth herein, the likelihood of air being introduced into the vascular system of the patient through use of the catheter system is decreased. It is understood the sequence(s) outlined in FIGS. 2A-2C can be varied without substantially deviating from the intent of the method(s) provided herein. Further, no limitations are intended by providing the specific embodiment(s) shown and/or described herein.

FIG. 2A is a simplified side view of a portion of one embodiment of the intravascular balloon catheter system 210. In this embodiment, the catheter system 210 includes the balloon catheter 218 which is at least partially, if not fully, submerged in a liquid solution 228. The liquid solution 228 can include water or another suitable solution.

In the embodiment illustrated in FIG. 2A, the balloon catheter 218 includes a guidewire 230, a guidewire lumen 232, an inflatable balloon 234, a catheter shaft 236 (illustrated in phantom in FIG. 2A) and a catheter sheath 238. In embodiments, the inflatable balloon 234 can be a double-balloon structure having an outer balloon and an inner balloon disposed and contained within the outer balloon. In the embodiment illustrated in FIG. 2A, the inflatable balloon 234 is shown in a preconditioned, deflated state. It is understood that the balloon catheter 218 can also include other structures which are not illustrated and/or described herein for ease in understanding.

During use, the balloon catheter 218 is positioned within the circulatory system (not shown) of the patient 12 (illustrated in FIG. 1). The guidewire 230 and guidewire lumen 232 are inserted into a pulmonary vein (not shown) of the patient 12, and the catheter shaft 236 and the inflatable balloon 234 are moved along the guidewire 230 and/or the guidewire lumen 232 to be positioned near an ostium (not shown) of the pulmonary vein.

In one embodiment, the catheter shaft 236, inflatable balloon 234, the guidewire lumen 232 and/or the guidewire 230 can all be retractable and/or extendable relative to the catheter sheath 238. For example, the catheter shaft 236, inflatable balloon 234, the guidewire lumen 232 and/or the guidewire 230 can initially be retracted into the catheter sheath 238 prior to use with the patient 12. In the embodiment illustrated in FIG. 2A, the catheter shaft 236, inflatable balloon 234, the guidewire lumen 232 and/or the guidewire 230 can be submerged in the liquid solution 228. As illustrated in FIG. 2A, at least portions of the catheter shaft 236, the inflatable balloon 234, the guidewire lumen 232 and/or the guidewire 230 can then be moved longitudinally in a direction (illustrated by arrow 240) to emerge from the catheter sheath 238 while submerged in the liquid solution 228. At this point, one or more bubbles 242 may be present on an exterior surface 244 of the inflatable balloon 234. In the deflated state, the exterior surface 244 of the inflatable balloon 234 may be somewhat creased, folded and/or pleated, which can facilitate “hiding” or positioning of bubbles 242 on or near the exterior surface 244. In an alternative embodiment, at least portions of the catheter shaft 236, the inflatable balloon 234 (in the deflated state), the guidewire lumen 232 and/or the guidewire 230 can be moved longitudinally in a direction (illustrated by arrow 240) to emerge from the catheter sheath 238 prior to being submerged in the liquid solution 228.

FIG. 2B is a simplified side view of the portion of the intravascular balloon catheter system 210 illustrated in FIG. 2A. In the embodiment illustrated in FIG. 2B, the catheter system 210 includes the inflatable balloon 234 shown in a conditioned, inflated state, and submerged in the liquid solution 228. In this embodiment, inflation of the inflatable balloon 234 can occur while the inflatable balloon 234 is submerged in the liquid solution 228. In the inflated state, the exterior surface 244 of the inflatable balloon 234 is substantially smooth and without any significant creases or folds. In one such embodiment, the bubbles 242 which were positioned on or near the exterior surface 244 of the inflatable balloon 234 can be wiped off or otherwise removed to leave the exterior surface 244 of the inflatable balloon 234 substantially devoid of bubbles 242. As used herein, the “conditioned state” means that the exterior surface 244 of the inflatable balloon 234 has been wiped substantially clean of any significant bubbles 242.

FIG. 2C is a simplified side view of the portion of the intravascular balloon catheter system 210 illustrated in FIG. 2A. In the embodiment illustrated in FIG. 2C, the catheter system 210 includes the inflatable balloon 234 shown in a conditioned, deflated state. In this embodiment, the previously inflated, inflatable balloon 234 has subsequently been deflated. In the conditioned state, the exterior surface 244 of the inflatable balloon 234 is substantially devoid of bubbles 242. In the embodiment illustrated in FIG. 2C, at least portions of the catheter shaft 236, the inflatable balloon 234, the guidewire lumen 232 and/or the guidewire 230 can then be moved longitudinally in a direction (illustrated by arrow 246) into the catheter sheath 238 while still submerged in the liquid solution 228. With the methods provided herein, the exterior surface 244 of the inflatable balloon 234 remains devoid of any substantial bubbles 242 (illustrated in FIG. 2A), while being positioned within the catheter sheath 238.

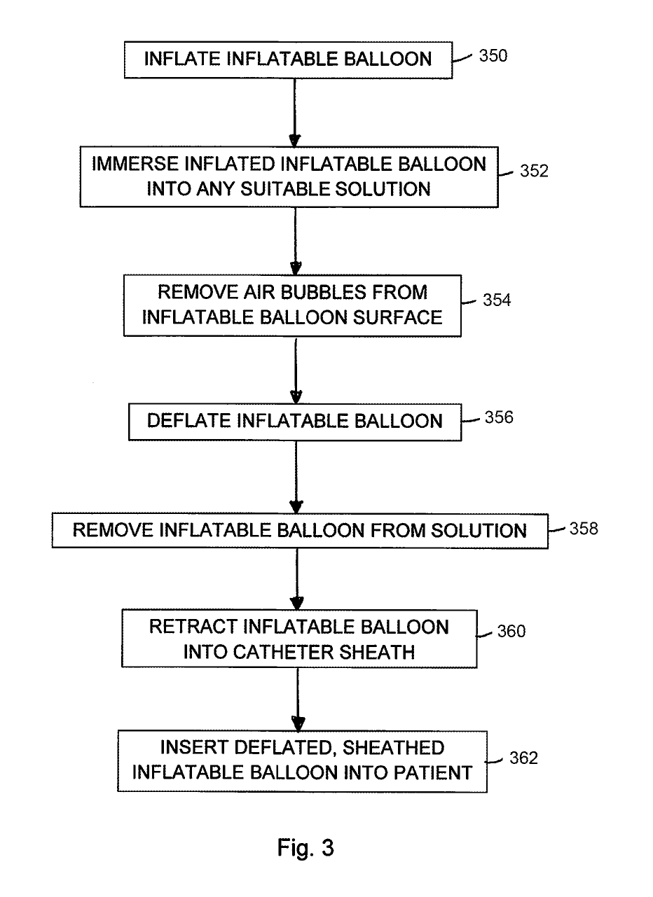

FIG. 3 is a flow chart providing one embodiment of a method for inhibiting the presence of air bubbles on the inflatable balloon of the intravascular balloon catheter system. It is understood that the method outlined in FIG. 3 can be altered without deviating from the intent and scope of the present disclosure. For example, the steps described and illustrated relative to FIG. 3 can have a different sequence from that illustrated in FIG. 3. Alternatively, steps can be added or omitted from those illustrated in FIG. 3.

At step 350, the inflatable balloon is inflated. Inflation of the inflatable balloon can occur either prior to or after submerging the inflatable balloon in a liquid solution, such as water.

At step 352, the inflated, inflatable balloon is submerged in the liquid solution. Alternatively, submerging the inflatable balloon in the liquid solution can occur prior to inflation of the inflatable balloon.

At step 354, air bubbles (or any other bubbles) are removed from the exterior surface of the inflatable balloon.

At step 356, the inflatable balloon is deflated.

At step 358, the deflated, inflatable balloon can be removed from the liquid solution.

At step 360, the deflated, inflatable balloon can be retracted into the catheter sheath. Alternatively, steps 358 and 360 can be reversed so that the deflated, inflatable balloon can be retracted into the catheter sheath prior to the deflated, inflatable balloon being removed from the liquid solution.

At step 362, the deflated, sheathed, inflatable balloon can be used inside of a patient.

It is understood that although a number of different embodiments of a method for inhibiting air bubbles on an inflatable balloon of an intravascular balloon catheter system have been illustrated and described herein, one or more features of any one embodiment can be combined with one or more features of one or more of the other embodiments, provided that such combination satisfies the intent of the present disclosure.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

I claim:

1. A method for conditioning an inflatable balloon of a balloon catheter prior to use inside a patient, the method comprising:
submerging the inflatable balloon in a liquid solution;
inflating the inflatable balloon;
removing any bubbles on an exterior surface of the inflated, inflatable balloon;
deflating the inflatable balloon while maintaining the inflatable balloon submerged in the liquid solution; and
retracting the deflated, inflatable balloon into a catheter sheath while maintaining the inflatable balloon submerged in the liquid solution.

2. The method of claim 1, further comprising removing the sheathed, deflated, inflatable balloon from the liquid solution.

3. The method of claim 2, wherein submerging the inflatable balloon in the liquid solution includes submerging the inflatable balloon in the liquid solution with the balloon in a deflated state, and wherein inflating the inflatable balloon is performed after submerging the inflatable balloon in the liquid solution.

4. The method of claim 2, wherein inflating the inflatable balloon is performed prior to submerging the inflatable balloon in the liquid solution.

5. The method of claim 2, wherein the balloon catheter is a cryoablation catheter, and wherein the inflatable balloon is configured to receive a cryogenic fluid.

6. The method of claim 5, wherein the inflatable balloon is a double-balloon structure having an outer balloon and an inner balloon disposed and contained within the outer balloon.

7. The method of claim 6, further comprising performing a cryoablation procedure after removing the sheathed, deflated, inflatable balloon from the liquid solution.

8. A method for conditioning an inflatable balloon of a balloon catheter prior to use inside a patient, the method comprising the steps of:
positioning the balloon catheter within a catheter sheath such that the inflatable balloon extends from an end of the catheter sheath;
submerging the end of the catheter sheath and the inflatable balloon in a liquid solution;
inflating the inflatable balloon;
removing any bubbles on an exterior surface of the inflated, inflatable balloon;
deflating the inflatable balloon; and
retracting the deflated, inflatable balloon into the catheter sheath while maintaining the inflatable balloon, the guidewire lumen and the end of the catheter sheath submerged in the liquid solution.

9. The method of claim 8, further comprising removing the catheter sheath and the deflated, inflatable balloon from the liquid solution.

10. The method of claim 9, wherein submerging end of the catheter sheath and the inflatable balloon in the liquid solution is performed with the inflatable balloon in a deflated state, and wherein inflating the inflatable balloon is performed after submerging the inflatable balloon in the liquid solution.

11. The method of claim 9, wherein inflating the inflatable balloon is performed prior to submerging the end of the catheter sheath and the inflatable balloon in the liquid solution.

12. The method of claim 9, wherein the balloon catheter is a cryoablation catheter, and wherein the inflatable balloon is configured to receive a cryogenic fluid.

13. The method of claim 12, wherein the inflatable balloon is a double-balloon structure having an outer balloon and an inner balloon disposed and contained within the outer balloon.

14. The method of claim 13, further comprising performing a cryoablation procedure with the balloon catheter after removing the catheter sheath and the inflatable balloon from the liquid solution.

15. A method for conditioning an inflatable balloon of a balloon catheter prior to use inside a patient, the balloon catheter including a guidewire lumen, the method comprising the steps of:
disposing the balloon catheter within a catheter sheath, with the inflatable balloon in a deflated state;
submerging an end of the catheter sheath, the inflatable balloon and the guidewire lumen in a liquid solution;
moving the balloon catheter longitudinally relative to the catheter sheath so that the inflatable balloon and the guidewire lumen extend from the end of the catheter sheath;
inflating the inflatable balloon;
removing any bubbles on an exterior surface of the inflated, inflatable balloon;
deflating the inflatable balloon; and
retracting the deflated, inflatable balloon and the guidewire lumen into the catheter sheath while maintaining the inflatable balloon, the guidewire lumen and the end of the catheter sheath submerged in the liquid solution.

16. The method of claim 15, further comprising removing the catheter sheath and the deflated, inflatable balloon from the liquid solution.

17. The method of claim 16, wherein the balloon catheter is a cryoablation catheter, and wherein the inflatable balloon is configured to receive a cryogenic fluid.

18. The method of claim 17, wherein the inflatable balloon is a double-balloon structure having an outer balloon and an inner balloon disposed and contained within the outer balloon.

19. The method of claim 16, further comprising disposing a guidewire within the guidewire lumen prior to submerging the end of the catheter sheath, the inflatable balloon and the guidewire lumen in the liquid solution.

20. The method of claim 16, further comprising performing a cryoablation procedure with the balloon catheter after removing the catheter sheath and the inflatable balloon from the liquid solution.
